(19) United States
(12) Patent Application Publication
(10) Pub. No.: US 2026/0175082 A1
(43) Pub. Date: Jun. 25, 2026

(54) Leveraging Large-Scale Exercise Device Metrics to Provide Personalized Experiences

(71) Applicant: Echelon Fitness Mulitmedia LLC, Chattanooga, TN (US)

(72) Inventors: Lou Lentine, Chattanooga, TN (US); John Santo, Chattanooga, TN (US)

(73) Assignee: Echelon Fitness Mulitmedia LLC, Chattanooga, TN (US)

(21) Appl. No.: 18/729,067

(22) PCT Filed: Feb. 13, 2023
(86) PCT No.: PCT/US2023/012917

§ 371 Date: Jul. 15, 2024

Related U.S. Application Data
(60) Provisional application No. 63/310,746, filed on Feb. 16, 2022.

(51) Int. Cl. A63B 24/00; A61B 5/11; A63B 22/02; G06N 20/00; G06Q 30/02; G06Q 30/0601; G16H 40/63

(52) U.S. Cl. CPC A63B 24/0062; A61B 5/1118; A63B 22/02; G06N 20/00; G06Q 30/02; G06Q 30/0631; G16H 40/63; A63B 2225/20

(57) ABSTRACT

In some aspects, the techniques described herein relate to a method including: collecting user data relating to a user's use of a fitness device; optionally capturing images of the user; building a profile for the user; augmenting the profile with interaction data; inputting the profile to a machine learning model, the machine learning model generating a classification; and outputting the classification.

### BACKGROUND

Currently, many network-enabled exercise devices provide streaming media as incentives to perform activities. For example, an exercise bike or spin bike can include a display that presents classes or other video content while a user engages in a physical activity. Leaderboards can be used to encourage competition while monitoring the user's performance. However, such exercise devices currently do not leverage additional data extrinsic to fitness data to provide improved recommendations to users.

### BRIEF DESCRIPTION OF THE DRAWINGS

FIG. 1 is a block diagram of a system for providing fitness activities and personalized recommendations to a user according to some of the example embodiments.

FIG. 2 is a flow diagram illustrating a method for generating a recommendation based on fitness data according to some of the example embodiments.

FIG. 3 is a flow diagram illustrating a method for generating a fitness plan according to some of the example embodiments.

FIG. 4 is a flow diagram illustrating a method for generating content recommendations based on sign on data according to some of the example embodiments.

FIG. 5 is a block diagram of a computing device according to some embodiments of the disclosure.

### DETAILED DESCRIPTION

In some aspects, the techniques described herein relate to a method including: collecting user data relating to a user's use of a fitness device; optionally capturing images of the user; building a profile for the user; augmenting the profile with interaction data; inputting the profile to a machine learning model, the machine learning model generating a classification; and outputting the classification.

In some aspects, the techniques described herein relate to a method including: performing, via a fitness device, a body scan of the user, the body scan including a three-dimensional representation of the user; receiving, via a fitness device, fitness goals, the fitness goals entered by the user or synthesized from the body scan; validating the fitness goals based on at least the body scan to generated validated fitness goals; and generating a customized fitness plan based on the validated fitness goals.

In some aspects, the techniques described herein relate to a method including: authenticating, by a user of a fitness device, with an external service; merging data managed by the external service with fitness data associated with the user to generate merged data; classifying the merged data to generate a label for the user; receiving a content item responsive to a query including the label; and displaying the content item to the user.

In some aspects, the techniques described herein relate to a non-transitory computer readable storage medium for tangibly storing computer program instructions capable of being executed by a computer processor, the computer program instructions defining the steps of the foregoing methods.

In some aspects, the techniques described herein relate to a computer system including a remote platform and an exercise device, the system configured to perform the foregoing methods.

### 1 ML-Based Personalized Recommendations

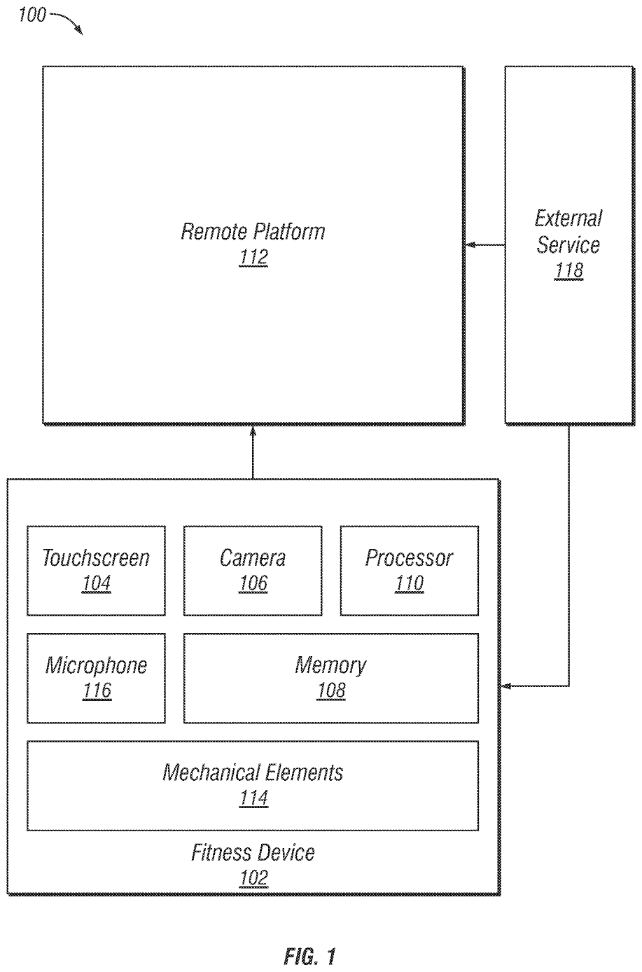

FIG. 1 is a block diagram of a system for providing fitness activities and personalized recommendations to a user according to some of the example embodiments. FIG. 2 is a flow diagram illustrating a method for generating a recommendation based on fitness data according to some of the example embodiments.

In general, the example embodiments provided machine learning (ML) or artificial intelligence (AI) recommendations to users based on fitness data entered by users or estimated based on captured data regarding the users.

In some embodiments, the system 100 includes an exercise device 102 that can be used to collect user data (step 202). For example, in an embodiment, a touchscreen 104 can be provided that allows a user to set fitness goals (e.g., age, weight loss goals, exercise duration/frequency goals, etc.). In some embodiments, the user can input other types of data such as health conditions, injuries, etc. In some embodiments, the data can be recorded from sensors on the user (e.g., heart rate monitors, smart watches, etc.).

Alternatively, or in conjunction with the foregoing, the exercise device 102 can include a camera 106. In some embodiments, the camera 106 can record an image (or a video comprising image frames) of the user (step 204). In some embodiments, the image can be input and processed (step 206) by a convolutional neural network (CNN) stored in memory 108 and executed by a processor 110 to classify (step 208) features of the user (e.g., the weight of the user, the height of the user, the age of the user, the strength or fitness tone of a user, injuries, health issues, etc.). In some embodiments, multiple CNNs can be used while in other embodiments, different types of image processing models can be used. Alternatively, or in conjunction with the foregoing, the exercise device 102 can include a reflective display screen (e.g., mirrored screen) (not illustrated) allowing the user to see themselves while the exercise device 102 is collecting data. In some embodiments, the reflective display screen can simultaneously display computer graphics while reflecting the user and can provide augmented displays over the reflection of the user. While the embodiments describe the processing of image data on exercise device 102, in some embodiments, the processing of images can be performed by remote platform 112.

Alternatively, or in conjunction with the foregoing, the exercise device 102 can comprise an exercise bike, spin bike, rowing machine, treadmill or similar device. In these embodiments, the user data can include performance metrics including aggregated metrics (e.g., average speed, average resistance, average stroke rate, etc.) as well as instantaneous metrics recorded by the device. In some embodiments the exercise device 102 may include mechanical elements 114. The mechanical elements of exercise device 102 are not limiting, and various different types of exercise devices may include different mechanical elements. For example, a spin or exercise bike may include a flywheel or types of resistance elements. A rowing machine may include a fan or other type of resistance element. A treadmill may include a motor or similar device. Mechanical elements 114 may include additional elements such as physical controls (e.g., handlebars), structural elements, or other types of physical devices. While the following embodiments describe selected physical elements in more detail, any such discussion is not intended to be limiting.

In some embodiments, the exercise device 102 can be installed in a user's home. In other embodiments, the exercise device 102 can be installed in a public or semi-public location (e.g., retail store, gym, etc.)

In some embodiments, each of the above data points can be recorded and associated with a given user into a profile or other data structure (step 210). In some embodiments, the profile can be augmented with data regarding objects a user interacts with (referred to as interaction data) (step 212). Examples of objects include digital content items (e.g., fitness classes), products purchased on e-commerce websites or mobile applications, etc. Generally, any object capable of a digital representation can be considered an object that a user can interact with, and the disclosure does not limit the embodiments as such. In some embodiments, the recorded user data can be combined interaction data to form a training dataset used to train (step 214) the model. In some embodiments, the predicted output comprises interaction data while the input data comprises the user data. In some embodiments, the system can also use an outcome as a label (positive or negative). For example, if a user's goal is to lose weight, the system can use the current weight of the user to determine if the user met that goal. This comparison can then be added to the label to ensure that users who meet goals are identified as positive examples in the system. In some embodiments, the system can use this goal-meeting criteria to exclude users from training who do not meet their goals. That is, the training corpus can comprise all users who meet goals (or otherwise satisfy a gating criteria). In another embodiment, the meeting or not meeting of goals can be added to the predicted output.

In some embodiments, the profile can be converted into a vector or other processible data structure for use in training an ML/AI model. Various types of models can be trained to perform prediction. In an embodiment, a collaborative-based filtering approach can be used to recommend interaction data to users. In some embodiments, the interaction data can include recommending content to interact with such as food, clothing, supplements, recipes, etc. In another embodiment, a deep neural network (DNN) can be trained to predict interaction data for users. In this embodiment, the training data can be processed to generate examples. Each example includes user data for a given user and the output vector comprises an item of interaction data. For example, a user's fitness goals can comprise the input and the output can comprise a purchase on an e-commerce website. In this manner, a single user can be associated with multiple items of interaction data, increasing the training data size. In some embodiments, a time-based threshold can be used to filter the training data. For example, the last six months of interactions may only be considered. In other embodiments, the time-based threshold can be based on the changing of user data. For example, the next six months after the user updated their fitness goals can be used as the window for interaction data. Thus, if the user updates their weight goal, the system can only analyze interactions (e.g., purchases) the user makes after making that change. In some embodiments, previous user data can be processed independently. Thus, a single user can have a first profile and a second profile, with corresponding time-based periods for generating examples from interaction data. In some embodiments, a recurrent neural network (RNN) such as a long short-term memory (LSTM) or gated recurrent unit (GRU) can be used. In such an embodiment, a series of interactions can be processed in a single window for each user. That is, a given sample of user data (e.g., weight goal) can be used to select a fixed period of interaction data (e.g., the next three months of interactions) and those interactions can be batched as the training data for a given sample of user data. The use of an RNN can thus factor time-based trends of a user. Other models using the above techniques can be employed such as random forest (RF) designs, gradient booting model (GBM) architectures, etc.

Alternatively, or in conjunction with the foregoing, the ML/AI model used can be used to classify a user based on the user data. For example, in some embodiments, the model can be used to classify an experience, competitiveness, etc. of a user. In some embodiments, the training data for such a model can comprise the user data and a derived label describing the category of the user. In some embodiments, a series of rules can be used to classify users in an unsupervised manner. For example, users can be classified as “competitive” if they perform a number of workouts above a first threshold, “casual” if the perform a number of workouts below the first threshold but over a second threshold, and “inactive” if the perform a number of workouts below the second threshold. The same type of rules-based approach can be applied to other metrics recorded by an exercise device 102. Thus, a user can be categorized and then their user data (e.g., weight goals, activity goals) can be used to predict (step 216) a category for future users. In some embodiments, a clustering routine can be used to cluster users and then manually label the clusters. For example, a k-means clustering routine can be used to cluster users based on user data and human editors can label each cluster.

The foregoing embodiments can provide significant benefits over existing techniques. For example, many existing techniques rely solely on matching users to one another based on demographic data or based interactions. For example, some systems recommend exercise devices to users within a certain age range, excluding others. Other systems record user interactions with one type of exercise device and provide recommendations to purchase the same type of exercise device.

By contrast, the foregoing embodiments provide highly relevant recommendations not just on the features of the users themselves, but on their desired and accomplished behavior. For example, a given user may be fifty-five years old, but may want to lose a certain amount of weight while also building strength. In existing systems, this profile would be matched with similar users (e.g., same age, same goals, etc.) but such systems do not consider if similar users met their goals. Thus, the interactions of a user who failed to meet a goal are not as relevant as the interactions of a user who did meet their goals. The example embodiments remedy this problem by explicitly considering goal targets and whether users meet these targets when training the predictive model.

### 2 Holistic Health Monitoring

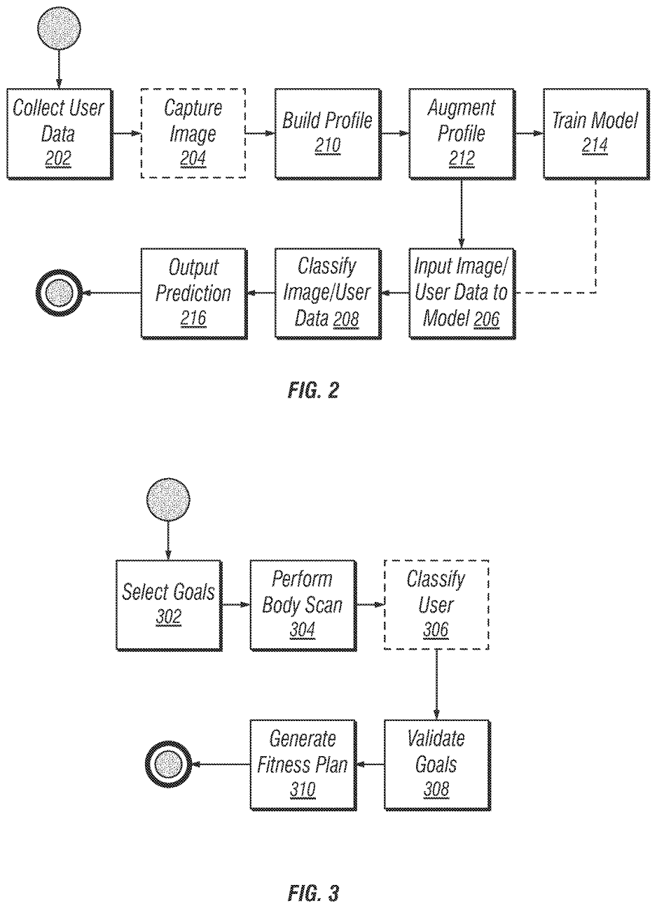

FIG. 3 is a flow diagram illustrating a method for generating a fitness plan according to some of the example embodiments.

In an embodiment, the system 100 can further be configured to provide holistic fitness recommendations for users based on captured user data. In an embodiment, an exercise device 102 is equipped with a display device (e.g., touchscreen 104), camera 106, or other image capture device. In some embodiments, the display device can display a plurality of user interfaces allowing the user to setup a fitness plan or routine. For example, a fitness plan can comprise a thirty-day schedule of fitness activities that a user desired to perform to meet a goal (e.g., weight loss). The specific duration is not limiting and, indeed, can change as will be discussed.

During an initialization process, the user can select (step 302) one or more goals via a first user interface. For example, the user can set a target weight (and provide a current weight), set a target distance to travel, set a target average speed or heartrate, or set a target based on any other measurable data point. In some embodiments, the user can specify multiple goals, and the disclosure is not limited to a single goal.

After a user selects one or more goals, the exercise device 102 transitions to a second user interface. In this interface, the exercise device 102 enables the camera 106 or other image capture device. The exercise device 102 can then present instructions via the interface that instructs a user to slowly rotate while remaining in a fixed position from the camera 106. While the user rotates, the exercise device 102 can capture image of the user. From these images, the exercise device 102 can build a three-dimensional model of the user (step 304). In some embodiments, the three-dimensional model can comprise a point cloud representing the user's body. In some embodiments the exercise device 102 can include a Lidar sensor to aid in building the three-dimensional model. In some embodiments, the point cloud can be used to classify (step 306) aspects of the user including, for example, a weight class, overall fitness level, strength level of individual muscles/body parts, etc. In some embodiments, additional measured data can be used supplement classified data. For example, if a user weights themselves using a scale communicatively coupled to the system, the measured weight can be used in lieu of a predicted weight class. In other embodiments, it can be used to check the predicted results.

Using the classification and the goals, the exercise device 102 can then validate the goals (step 308). In some embodiments, the exercise device 102 can first confirm if the specified goals are safe. For example, a goal of losing one hundred pounds in one month may be rejected as being a health risk. Alternatively, or in conjunction with the foregoing, the exercise device 102 can confirm if the goals are capable of being met. For example, a goal of running one million miles in one month may be rejected as being impossible. Alternatively, or in conjunction with the foregoing, the system can validate a goal based on a body scan. For example, the system can classify the user as athletic or non-athletic and can use this classification to determine if an objectively possible fitness goal is in fact possible based on the user's body classification. For example, a user who is physically fit may be capable of meeting a higher distance goal than a user who is not physically fit.

In some embodiments, the foregoing functionality can exclude the use of manual user input (e.g., goals) and can rely solely on a graphical interface. For example, in some embodiments, the system can perform a body scan and present an image of the user (either actual or virtual). The system can then allow the user to manipulate the three-dimensional representation of themselves. In some embodiments, the user can manipulate their model to mimic a goal (e.g., weight loss, strength training etc.). In other embodiments, the user can still input a goal and the system can set a weight goal and the system can adjust the three-dimensional body model based on the difference between the scanned model and the target weight goal. In some embodiments, the input can be voice-activated. For example, a user can give specific voice commands (“I want to lose 15 pounds”) or more general voice commands (“I want to lose weight” or “I'd like to feel better”) via a microphone 116 and the system can update the three-dimensional model based on the voice commands (e.g., after converting to text representations). In some embodiments, the use of a three-dimensional scan of the actual user, and adjustments to the model, allows a user to more intuitively visualize a goal. Further, in some embodiments, invalid goals (discussed above) can be temporarily applied to the model to illustrate the negative impact of the goals.

Once the user's goals are validated in the above manner, the system can generate a fitness plan (step 310). In some embodiments, the user can manually specify a fitness plan criterion (e.g., duration) that can be validated in a similar manner as described above. In other embodiments, the system can set a fitness plan parameter based on the validated goals. For example, if the user's goal is to lose one hundred pounds in a month the system can either adjust the duration (e.g., one year) or adjust the goal (e.g., to eight pounds). In some embodiments, specific rules can be used (e.g., as promulgated by health organizations) can be used to adjust fitness plan durations or goals. In some embodiments, the adjusted fitness plan can be persistently saved to a user's account and can be used to recommend activities. For example, if the user is recommended a two-month plan to lose twenty pounds, a series of weekly activities can be recommended to the user to complete the plan. In some embodiments, the system can continuously monitor the user's progress toward the goal and adjust the goal as described above. In other embodiments, human fitness instructors can be used to adjust the goals. For example, a customized fitness plan created by the system can be reviewed by a fitness instructor which can manually adjust the plan as needed. In some embodiments, the system can allow for bidirectional communication between instructors and users. For example, an instructor can send a communication to a user after viewing their fitness plan to encourage, suggest changes, or invite them to specific events. In some embodiments, the system can provide various gamification features such as goals, awards, or similar mechanisms to further encourage users to adhere to the customized fitness plan.

In some embodiments, the above functionality can be continuously executing in a passive manner. For example, if the user uses a mirror exercise device, the above process can be run continuously (i.e., periodic body scans) and used to adjust the fitness plan based on the user's performance. In some embodiments, the system can also receive fitness metrics to intuit the progress of a user. For example, if the user's goal is to increase strength, the system can monitor an average resistance or speed of the user when using an exercise device and determine their progress, completion velocity, etc.

### 3 Leveraging External Data During Sign On

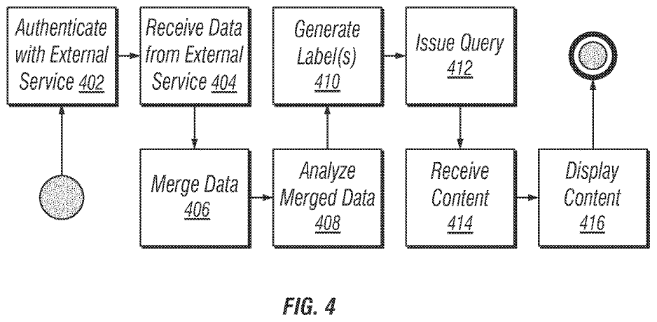

FIG. 4 is a flow diagram illustrating a method for generating content recommendations based on sign on data according to some of the example embodiments.

In the various embodiments, users may be required to authenticate to the system. For example, prior to using an exercise device 102 (or mobile application), a user may be required to login to the system. In most existing devices, a user can authenticate directly with the system 100 itself. However, as illustrated, in the system 100, users may authenticate (step 402) via an external service 118. In some embodiments, the external service 118 can comprise an e-commerce platform or similar entity. In some embodiments, during the sign on process, the system 100 (e.g., remote platform 112) can receive data (step 404) stored by the e-commerce platform and can merge (step 406) this information with the profile stored by the systems. Alternatively, or in conjunction with the foregoing, the system 100 can provide data it records during fitness activities to the e-commerce system (which can, in turn, merge the fitness data with its e-commerce data).

In some embodiments, the system 100 can analyze (step 408) a user's data stored by the external service and provide recommended content based on the data stored by the external system and the user's fitness data. In some embodiments, the user's fitness data can be used as an input to an ML model that can generate one or more labels (step 410) (e.g., using a multi-label classifier) that represent a user's likely interests. The system can then issue a query (step 412) to the external service to receive content items (step 414) relevant to the predicted labels. In some embodiments, the system 100 can then display (step 416) the content items on the touchscreen 104 or other display of the exercise device 102. For example, if a user frequently joins yoga classes, the system can input the user's history of activities to the multi-label classifier and will likely receive a label (“yoga”) representing the user's interests. The system can then provide this label to the external service for recommended content items (e.g., yoga pants, mats, books, etc.). As another example, the multi-label classifier can be trained to predict a category of the user along a more generic spectrum (e.g., from inactive to athletic). This classification can then be used to determine whether to query the external service for supplements (e.g., for athletic users) or healthy groceries (e.g., for inactive users).

### 4 Group Dynamics in Fitness Systems

In general, the system 100 can allow for multiple users to participate in fitness activities. In some embodiments, the system 100 can allow users to form groups of users and can enable group-specific actions on such groups, as discussed in more detail below.

In an embodiment, the system 100 can allow one or more users to join a group. In some embodiments, a first user can initiate a group and the first user can transmit invitations to other users, thereby allowing other users to join. In other embodiments, users can search for groups and join groups of interest. In other embodiments, the system 100 can recommend groups by matching users to groups. For example, the system 100 can use an ML model to predict the affinity of a given user to groups managed by the system 100.

In an embodiment, the system 100 can allow users within a group to create challenges. In some embodiments, a challenge can comprise a fitness plan or one or more fitness goals (as discussed above). In some embodiments, the challenges can be time-bound (e.g., lasting one month). In some embodiments, each challenge can include gamification elements (e.g., rewards, badges, etc.). In some embodiments, the gamification elements can be designed by users of the group, enabling custom gamification elements only available to group members. In some embodiments, when users complete a group challenge, the system 100 can assign the custom gamification element to the users for display on a profile screen for the user. In some embodiments, a given group challenge can include a monetary award. In some embodiments, this award can be an amount shared among all users that complete a group challenge. In other embodiments, the award can be a donation or similar charitable award.

In some embodiments, the system 100 can additionally publish challenge schedule data and attendance or progress data (collectively, accountability data). In some embodiments, the system 100 can broadcast each user's accountability data for any given challenge to all other users. In some embodiments, the accountability data of a user can be transmitted to the user or other users as a push notification.

In some embodiments, other users not in a group can participate in a group challenge or group activity. For example, fitness instructors can join a group challenge or group activity. In some embodiments, the system 100 can select a fitness instructor to join a group challenge or group activity based on the interests of the group users (e.g., select a fitness instructor most users prefer). In some embodiments, the system 100 can encourage instructors to communicate with group users. For example, when a user of the group records a personal best (e.g., time, speed) for a fitness activity, the system 100 can detect such an event and encourage a fitness instructor (e.g., a favorite instructor) to congratulate the user.

In some embodiments, the system 100 can further facilitate intra-group communications. In some embodiments, the system 100 can monitor fitness data of the users in the group and determine when to encourage users to communicate. For example, when a user of the group records a personal best (e.g., time, speed) for a fitness activity, the system 100 can detect such an event and encourage other group members to congratulate the user. In some embodiments, during group activities (i.e., fitness activities that can only be participated in by users of a group), the system 100 can enable a voice chat feature allowing the users to talk to each other during the group activity. In some embodiments, the voice chat feature can be constant (that is, users can talk at any point during the activity). In other embodiments, the system 100 can enable a “tap to talk” feature allowing users to be muted until they wish to talk. In some embodiments, the system 100 can provide both group talk features as well as person-to-person talk features.

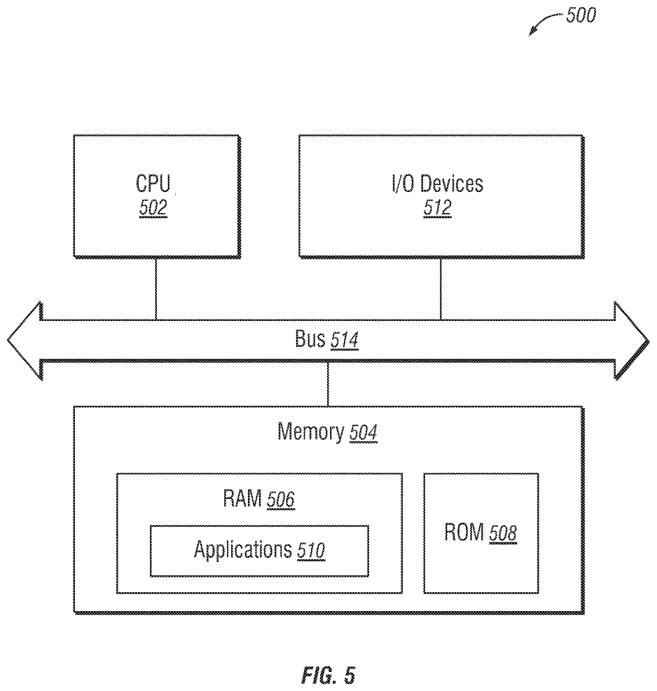

FIG. 5 is a block diagram of a computing device according to some embodiments of the disclosure.

As illustrated, the device 500 includes a processor or central processing unit (CPU) such as CPU 502 in communication with a memory 504 via a bus 514. The device also includes one or more input/output (I/O) or peripheral devices 512. Examples of peripheral devices include, but are not limited to, network interfaces, audio interfaces, display devices, keypads, mice, keyboard, touch screens, illuminators, haptic interfaces, global positioning system (GPS) receivers, cameras, or other optical, thermal, or electromagnetic sensors.

In some embodiments, the CPU 502 may comprise a general-purpose CPU. The CPU 502 may comprise a single-core or multiple-core CPU. The CPU 502 may comprise a system-on-a-chip (SoC) or a similar embedded system. In some embodiments, a graphics processing unit (GPU) may be used in place of, or in combination with, a CPU 502. Memory 504 may comprise a non-transitory memory system including a dynamic random-access memory (DRAM), static random-access memory (SRAM), Flash (e.g., NAND Flash), or combinations thereof. In one embodiment, bus 514 may comprise a Peripheral Component Interconnect Express (PCIe) bus. In some embodiments, bus 514 may comprise multiple busses instead of a single bus.

Memory 504 illustrates an example of non-transitory computer storage media for the storage of information such as computer-readable instructions, data structures, program modules, or other data. Memory 504 can store a basic input/output system (BIOS) in read-only memory (ROM), such as ROM 508, for controlling the low-level operation of the device. The memory can also store an operating system in random-access memory (RAM) for controlling the operation of the device

Applications 510 may include computer-executable instructions which, when executed by the device, perform any of the methods (or portions of the methods) described previously in the description of the preceding Figures. In some embodiments, the software or programs implementing the method embodiments can be read from a hard disk drive (not illustrated) and temporarily stored in RAM 506 by CPU 502. CPU 502 may then read the software or data from RAM 506, process them, and store them in RAM 506 again.

The device may optionally communicate with a base station (not shown) or directly with another computing device. One or more network interfaces in peripheral devices 512 are sometimes referred to as a transceiver, transceiving device, or network interface card (NIC).

An audio interface in peripheral devices 512 produces and receives audio signals such as the sound of a human voice. For example, an audio interface may be coupled to a speaker and microphone (not shown) to enable telecommunication with others or generate an audio acknowledgment for some action. Displays in peripheral devices 512 may comprise liquid crystal display (LCD), gas plasma, light-emitting diode (LED), or any other type of display device used with a computing device. A display may also include a touch-sensitive screen arranged to receive input from an object such as a stylus or a digit from a human hand.

A keypad in peripheral devices 512 may comprise any input device arranged to receive input from a user. An illuminator in peripheral devices 512 may provide a status indication or provide light. The device can also comprise an input/output interface in peripheral devices 512 for communication with external devices, using communication technologies, such as USB, infrared, Bluetooth™, or the like. A haptic interface in peripheral devices 512 provides tactile feedback to a user of the client device.

A GPS receiver in peripheral devices 512 can determine the physical coordinates of the device on the surface of the Earth, which typically outputs a location as latitude and longitude values. A GPS receiver can also employ other geo-positioning mechanisms, including, but not limited to, triangulation, assisted GPS (AGPS), E-OTD, CI, SAI, ETA, BSS, or the like, to further determine the physical location of the device on the surface of the Earth. In one embodiment, however, the device may communicate through other components, providing other information that may be employed to determine the physical location of the device, including, for example, a media access control (MAC) address, Internet Protocol (IP) address, or the like.

The device may include more or fewer components than those shown in FIG. 5, depending on the deployment or usage of the device. For example, a server computing device, such as a rack-mounted server, may not include audio interfaces, displays, keypads, illuminators, haptic interfaces, Global Positioning System (GPS) receivers, or cameras/sensors. Some devices may include additional components not shown, such as graphics processing unit (GPU) devices, cryptographic co-processors, artificial intelligence (AI) accelerators, or other peripheral devices.

The subject matter disclosed above may, however, be embodied in a variety of different forms and, therefore, covered or claimed subject matter is intended to be construed as not being limited to any example embodiments set forth herein; example embodiments are provided merely to be illustrative. Likewise, reasonably broad scope for claimed or covered subject matter is intended. Among other things, for example, the subject matter may be embodied as methods, devices, components, or systems. Accordingly, embodiments may, for example, take the form of hardware, software, firmware, or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in an embodiment” as used herein does not necessarily refer to the same embodiment, and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms such as “and,” “or,” or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. Similarly, terms, such as “a,” “an,” or “the,” again, can be understood to convey a singular usage or to convey a plural usage, depending at least in part upon context. In addition, the term “based on” may be understood as not necessarily intended to convey an exclusive set of factors and may, instead, allow for the existence of additional factors not necessarily expressly described, again, depending at least in part on context.

The present disclosure is described with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general-purpose computer to alter its function as detailed herein, a special purpose computer, application-specific integrated circuit (ASIC), or other programmable data processing apparatus, such that the instructions, which execute via the processor of the computer or other programmable data processing apparatus, implement the functions/acts specified in the block diagrams or operational block or blocks. In some alternate implementations, the functions or acts noted in the blocks can occur out of the order noted in the operational illustrations. For example, two blocks shown in succession can, in fact, be executed substantially concurrently, or the blocks can sometimes be executed in the reverse order, depending upon the functionality or acts involved.

These computer program instructions can be provided to a processor of a general-purpose computer to alter its function to a special purpose; a special purpose computer; ASIC; or other programmable digital data processing apparatus, such that the instructions, which execute via the processor of the computer or other programmable data processing apparatus, implement the functions or acts specified in the block diagrams or operational block or blocks, thereby transforming their functionality in accordance with embodiments herein.

For the purposes of this disclosure, a computer-readable medium (or computer-readable storage medium) stores computer data, which data can include computer program code or instructions that are executable by a computer, in machine-readable form. By way of example, and not limitation, a computer-readable medium may comprise computer-readable storage media for tangible or fixed storage of data or communication media for transient interpretation of code-containing signals. Computer-readable storage media, as used herein, refers to physical or tangible storage (as opposed to signals) and includes without limitation volatile and non-volatile, removable, and non-removable media implemented in any method or technology for the tangible storage of information such as computer-readable instructions, data structures, program modules or other data. Computer-readable storage media includes, but is not limited to, RAM, ROM, EPROM, EEPROM, flash memory or other solid-state memory technology, CD-ROM, DVD, or other optical storage, magnetic cassettes, magnetic tape, magnetic disk storage, or other magnetic storage devices, or any other physical or material medium which can be used to tangibly store the desired information or data or instructions and which can be accessed by a computer or processor.

For the purposes of this disclosure, a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer-readable medium for execution by a processor. Modules may be integral to one or more servers or be loaded and executed by one or more servers. One or more modules may be grouped into an engine or an application.

Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than or more than all the features described herein are possible.

Functionality may also be, in whole or in part, distributed among multiple components, in manners now known or to become known. Thus, a myriad of software, hardware, and firmware combinations are possible in achieving the functions, features, interfaces, and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example to provide a complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

We claim:

1. A method comprising:
collecting user data relating to a user's use of a fitness device;
optionally capturing images of the user;
building a profile for the user;
augmenting the profile with interaction data;
inputting the profile to a machine learning model, the machine learning model generating a classification; and
outputting the classification.

2. A method comprising:
performing, via a fitness device, a body scan of a user, the body scan comprising a three-dimensional representation of the user;
receiving, via a fitness device, fitness goals, the fitness goals entered by the user or synthesized from the body scan;
validating the fitness goals based on at least the body scan to generated validated fitness goals; and
generating a customized fitness plan based on the validated fitness goals.

3. A method comprising:
authenticating, by a user of a fitness device, with an external service;
merging data managed by the external service with fitness data associated with the user to generate merged data;
classifying the merged data to generate a label for the user;
receiving a content item responsive to a query including the label; and
displaying the content item to the user.

4. A non-transitory computer readable storage medium for tangibly storing computer program instructions capable of being executed by a computer processor, the computer program instructions defining steps to execute the foregoing methods.

5. A computer system comprising a remote platform and an exercise device, the system configured to perform the foregoing methods.
